(19) United States
(12) Patent Application Publication
(10) Pub. No.: US 2010/0016843 A1
(43) Pub. Date: Jan. 21, 2010

(54) Tip Member for a Laser Emitting Device

(75) Inventors: Thomas Bragagna, Feldkirch (AT); Werner Graf, Schaffhausen (CH)

Correspondence Address: FISH & ASSOCIATES, PC;ROBERT D. FISH, 2603 Main Street, Suite 1000, Irvine, CA, 92614-6232, US

(73) Assignee: PANTEC BIOSOLUTIONS AG, Ruggell (LI)

(21) Appl. No.: 12/446,791

(22) PCT Filed: Oct. 25, 2007
(86) PCT No.: PCT/EP07/61502

§ 371 Date: Apr. 23, 2009

(30) Foreign Application Priority Data
Oct. 25, 2006 (EP) PCT/EP2006/067772

(51) Int. Cl. A61B 18/20

(52) U.S. Cl. 606/13

(57) ABSTRACT

A tip (8) comprising a first portion (8r) that is configured to be removably coupled to a laser porator (10) and a second portion (8h) defining an aperture (8x) and comprising a tissue biasing element (8a) that is configured to deform at least a portion (1a) of a tissue (1) that is subject to a laser treatment.

### FIELD OF THE INVENTION

This invention relates to a tip which is configured to be removably coupled to a laser porator, to facilitating poration of biological tissue, in particular the skin.

### BACKGROUND OF THE INVENTION

Document WO 03/047449 A1 discloses a tip which is configured to be removably coupled to a laser porator. The purpose of this tip is to protect the laser porator from contamination by debris. The debris may result when the laser porator emits a laser pulse onto the skin. The disadvantage of the laser porator as well as the tip disclosed is that they are only suitable for a single shot or single pulse laser system which is able to create a single ablation area on the skin. It is therefore not possible to create a plurality of pores in a single application of the laser porator. Further it is also not possible to reproduce the shape of create pores.

Numerous diseases and conditions involve skin in a direct or indirect manner, and most of the diseases and conditions are associated with or caused by an immunologic response to an exogenous stimulus. While an immunologic response is generally desirable in most instances (e.g., to combat infection), an auto- or alloimmune response is typically detrimental (e.g., in skin transplantation). Treatment of many skin diseases and conditions is often local and topical in response to an etiologic agent or stimulus (e.g., injury or infection). However, various other skin diseases and conditions are more diffuse in presentation and may be the result of regional agents or stimuli (e.g., exposure to allergen, radiation, etc.) and may in some cases even have systemic underlying conditions (e.g., histoincompatibility).

While topical treatment is often relatively simple and effective in many cases, treatment of diseases and conditions that manifest themselves in a relatively large area or have a systemic component is significantly more difficult. Conceptually the following option is available.

First, a drug may be applied over a relatively large-area (e.g., by application of topical ointment), but efficacy is often undesirable as the skin presents a permeability barrier to most compounds with molecular weight greater 500 Dalton. Even when the drug is a relatively small molecule, hydrophilic drugs will typically not be effectively delivered. Several strategies have been developed to circumvent at least some of these problems, including chemical approaches (e.g., micro/nanovesicle delivery via encapsulation into liposome and other lipophilic carriers; penetration enhancers, such as azone, alphahydroxy acids, etc) and mechanical approaches (e.g., partial removal of stratum corneum by tape stripping, dermabrasion, etc.). Unfortunately, most mechanical approaches are problematic or impractical, especially when a large area is to be treated, and many chemical approaches are not always well tolerated or difficult to formulate. Moreover, topically delivered dosages are often not high enough to achieve a desired effect.

Some attempts have also been made to improve transdermal delivery of higher volumes of a drug using a laser for puncturing the skin of a patient in a manner that does not result in bleeding. Such perforation typically penetrates through the stratum corneum or both the stratum corneum and the epidermis. This allows drug delivery through the skin. An example of such a laser, described in document EP 1133953 A1, provides one slit-shaped perforation with a width of up to 0.5 mm and a length of up to 2.5 mm. Unfortunately, the rate of drug delivery through such a perforation is limited. This perforation also provokes undesirable skin reactions and the perforation of the skin frequently causes pain. The perforation requires subsequent patch drug application. However, such administration of drugs often results in inconsistent drug dosages, inconvenient usage, and sometimes even in infections.

Therefore, while numerous methods of the administration of a drug into the skin or methods of the administration of a drug through the skin into the blood circulation of the human body are known in the art, all or almost all of them suffer from one or more disadvantages. In addition, while numerous methods for the administration of drugs through biological tissue, in particular through the skin are known in the art, all or almost all of them suffer from one or more disadvantages. Consequently, there is still a need to provide improved apparatuses, compositions and methods to improve drug delivery to the skin, the biological tissue and the human body for treatment of skin related conditions and for the treatment of various diseases and conditions.

### SUMMARY OF THE INVENTION

The inventors have now discovered that drugs can be safely and effectively applied to biological tissue, in particular an area of skin, in relatively high concentrations by creating a plurality of micropores with in particular predetermined geometry. More preferably, the pores will have a depth that is sufficient to create a channel in the stratum corneum of the skin to allow delivery of a drug to the epidermis, and more preferably to the epidermis and the dermis.

The object of the present invention is to improve the quality of the plurality of micropores created by a laser emitting device.

This problem is solved by a tip configured to be removably coupled to a laser porator and comprising the features of claim 1. Claims 2 to 22 disclose further advantageous tips. The problem is further solved with a laser porator comprising the features of claim 23. Claims 24 to 28 disclose further advantageous laser porators. The problem is further solved by a kit comprising a laser emitting device and a tip and comprising the features of claim 29.

The problem is in particular solved with a tip comprising a first portion that is configured to be removably coupled to a laser porator and a second portion that comprises a tissue biasing element that is configured to deform at least a portion of the tissue such as for example the skin that is within the opening of the tip, and that is subject to a laser treatment.

One advantage of the tip according to the invention is that it guarantees reproducible irradiation intensity on the tissue. A tip as known in the state of art would, if pressure is applied to the device and the tip laying on the tissue, cause the skin to camber into direction of the laser source, causing variable intensity of the laser beam hitting the tissue. This would lead to irreproducible pores, for example because the focal range of the laser is not in the area where the tissue is located, leading to the effect that some pores are not produced and/or some pores are produced with a not reproducible shape. This effect is avoided with the tip according to the invention.

The laser emitting device, also called laser porator, comprises a tip, preferably a disposable tip, through which the laser beam is projected onto the biological tissue such as for example the skin. The tip according to the invention includes a tissue biasing element that, upon contact with the tip, will force the tissue to be treated into a predetermined geometry, so the tissue below the tissue biasing element has a determined geometry.

Most typically, the predetermined geometry will ensure that the average distance between a laser mirror that steers the beam over the skin and the skin that is to be treated is substantially the same. Viewed from another perspective, the tissue biasing element will deform the skin such that the skin that is to be treated will be at the focal point of the laser beam throughout the area that is to be treated. The skin or other biological tissue will therefore be in a predetermined position with respect to the impacting laser beam. Such tissue biasing will allow for consistent application of a laser beam with known and/or uniform parameters onto the biological tissue.

Most preferably, the majority of micropores is dimensioned such that the pores allow administration of a drug to within the human body. To avoid bleeding the micropores are arranged such that the pores do not intersect with a (capillary) blood vessel. With respect to further micropore geometry, it should be appreciated that the side walls of micropores according to the inventive subject matter need not necessarily be straight. Indeed, it should be especially recognized that geometry of the wall of the micropores will have a significant influence on at least two factors that are critical for drug delivery: Among other things, the total inner surface can be easily modified by increasing the pore diameter and/or pore depth. Additionally, or alternatively, the wall angle may also deviate from a right angle (relative to the average surface of the stratum corneum), and as such will lead to an increase of total inner pore surface, and with that an increase in the potential area of drug delivery. Still further increases can be achieved by stepping the side walls of a pore. Second, as the micropores are subject to a healing/natural skin renewal process, the micropore geometry will also determine the time available for delivery of a drug across the micropore walls. Preferably, micropores generated with a porator as described below will not give rise to scarring, possibly due to photoablation and/or relatively small size of the pores.

It is further especially preferred that the laser of a laser porator is operated during pore formation in a q-switched or short pulsed mode and with pulse widths and energies such that laser irradiation will result in a blow-off effect without leading to coagulation. Thus, photoablation and/or photodisruption is particularly preferred. Such irradiation will typically vaporize the tissue with negligible creation of thermal damage. For example, suitable ranges of irradiance will be at least 10⁴ W/cm², and more preferably at least 10⁵ W/cm², even more preferably between 10⁵ W/cm² and 10⁹ W/cm², and most preferably between 10⁵ W/cm² and 10¹² W/cm² where energy doses of between about 0.01 J/cm² to 1000 J/cm², and more typically 0.1 J/cm² to 100 J/cm² are employed. Consequently, the laser pulse width/tissue exposure time is preferably less than 1 ms, more preferably less than 100 μs, even more preferably between 100 μs and 10 ns, and most preferably between 100 μs and 0.1 ps. Sizing and operation of lasers to achieve such parameters is well understood in the art, and many of the lasers and control systems therefore are commercially available. Consequently, and viewed from another perspective, it should be recognized that especially suitable operational parameters will be selected to provide a balance between minimum tissue damage and maximum desired effect.

The tip according to the invention allows the laser beam or the laser pulse to reproducible irradiate the tissue, in particular with reproducible intensity. This allows creating pores with high sophisticated properties. For example it may also be desirable to at least partially coagulate the pore walls A, and most typically the pore bottom 3e as exemplarily depicted in the left pore 2 in FIG. 16. The pore 2 to the left was first vertically formed in the skin 1 using q-switched or short pulsed laser mode to reduce thermal damage. The stratum corneum 1a, the epidermal layer 1b and the dermal layer 1c are shown. Preferably the laser is applied several times into the same pore 2 so that the lower end 3a, 3b, 3c, 3d of the pore 2 increases with each laser pulse applied. Subsequently, the bottom 3e of the formed pore 2 is coagulated using the laser in free-running mode (using the same laser at pulse widths of 100-1000 μs), or using another laser at a wavelength and fluence effective to coagulate. Thus, laser irradiation may also include a protocol in which a pore 2 is formed using the laser in a switched mode to operate under photoablative and/or photodisruptive conditions, and in which one or more laser pulses are applied with significantly longer duration. For example, the bottom 3e of the pore 2 (and/or the side walls A where desirable) may be irradiated with the same laser or a second laser or the same laser with a second wavelength or a second laser with a second wavelength in a non q-switched or short pulsed “free-running” mode to achieve at least partial coagulation. Due to the fact that the e.g. bottom 3e of the pore 2 is sealed, the diffusion B into the epidermal layer 1b can only be achieved through the e.g. walls A of the pores 2. This leads to a decelerated delivery of the drug to the systemic circulation in the dermal layer. Moreover, such partially sealed pores 2 will provide a horizontal drug delivery (i.e., parallel to surface of skin), which may delay the rate of delivery for at least some time. Of course, over time the coagulated tissue is repaired and accelerated delivery of the drug will then take place. The person of ordinary skill in the art will readily be able to determine a suitable time for irradiation to achieve coagulation, and it is generally contemplated that appropriate pulse widths will be in the range of about 10 μs to about 1000 μs, more preferably in the range of about 100 μs to about 500 μs.

In a further aspect of the inventive subject matter, it is desirable to create a plurality of pores 2 as disclosed in FIG. 16 to the right, without coagulated pore walls A, and it is desirable to create the pores 2 with reproducible shape. The pore 2 to the right allows a direct and preferably fast delivery of the drug to the systemic circulation in the dermal layer.

Thus, multiple applications of a drug through micropores in the same area are realized while maintaining a cosmetically and physiologically desirable environment. As applications as presented herein maintain adjacent tissue viability and structure, contemplated methods are also suitable for administration of a drug in large areas. It should further be especially appreciated that using contemplated methods, compositions, and devices will allow delivery of a high amount of a drug into a patient. Moreover, due to the control over drug delivery kinetic and dynamic via control of the pore geometry, drugs delivery can be personalized to accommodate different skin locations in a patient as well as different skin types among different patients. Similarly, drug delivery kinetic and dynamic can be tailored to a specific drug (e.g., slow delivery for fast acting drug, fast and high quantity delivery for instable drugs, etc.). Especially preferred porators suitable for the tips according to the invention, exemplary methods, and configurations are provided in the applicants' copending patent applications with the following serial numbers, all of which are incorporated by reference herein:

A micro-porator for porating a biological membrane to create a poration may be designed, for example, as the laser micro-porator disclosed in PCT patent application No. PCT/EP2006/061639 of the same applicant, and entitled “Laser microporator and method for operating a laser microporator”.

The biological membrane may be porated according to a method, for example, as disclosed in PCT patent application No. PCT/EP2005/051703 of the same applicant, and entitled “Method for creating a permeation surface”.

A micro-porator for porating a biological membrane and an integrated permeant administering system may be designed, for example, as the microporator disclosed in PCT patent application No. PCT/EP2005/051702 of the same applicant, and entitled “Microporator for porating a biological membrane and integrated permeant administering system”.

A system for transmembrane administration of a permeant and a method for administering a permeant may be designed, for example, as the system disclosed in PCT patent application No. PCT/EP2006/050574 of the same applicant, and entitled “A system for transmembrane administration of a permeant and method for administering a permeant”.

A transdermal delivery system for administration of a drug and a method for administering the drug may be designed, for example, as the system disclosed in PCT patent application No. PCT/EP2006/067159 of the same applicant, and entitled “Transdermal delivery system and method for treating infertility”.

Thus, in one preferred aspect, the inventors contemplate a tip and a laser porator for porating a biological tissue such as the skin, for example for treating a skin related disease or disorder in which an area of porated skin is formed and wherein the area comprises a plurality of pores. Most typically, the area is equal or greater than 1 cm², more typically equal or greater than 10 cm², even more typically equal or greater than 25 cm² or greater than 100 cm². The number of pores may vary considerably, and suitable numbers include those in the range of between about 10-100,000. However, and especially where large areas are treated, higher numbers are also contemplated. Therefore, the number of pores/cm² may generally vary between about 1-10, more typically 10-100, or 100-1000, and in rare cases even higher. Similarly, the pattern of pores in the skin may vary as well, and isotropic distribution is generally preferred. However, and especially where anatomically and/or physiologically advisable, anisotropic distribution is also contemplated. For example, areas of relatively slow drug diffusion (e.g., fibrotic tissue, thick epidermis, thick stratum corneum, etc.) may have a higher number of pores, whereas other areas may have less. Similarly, areas with disease focus may concentrate the pores in the focus and reduce the number of pores in the periphery. Similarly, areas that require a high dosage or volume of the drug may have a higher density in pores than those that require a lower dosage. In another preferred aspect, the inventors contemplate a tip and a laser porator for porating a biological tissue such as the skin, for example for delivering a high amount of a drug within a small area of for example less than 1 cm².

It is generally preferred that at least some of the pores have a predetermined geometry that is at least in part a function of the drug. Moreover, the predetermined geometry will preferably control the inner pore surface area, the time to pore re-closure, and/or the pore depth (i.e., layer of epidermis or dermis that is contacted with the drug). The drug (or drugs) is then applied to the area of porated skin, which may be done in single, repeated, or continuous (e.g., under occlusion) manner. While numerous alternative wavelengths are deemed suitable, particularly preferred wavelengths for laser ablation is at a wavelength of at least 2500 nm, and most preferably at about 2950 nm.

The following figures and description will provide sufficient guidance to a person of ordinary skill in the art to make and use the tips contemplated herein.

### BRIEF DESCRIPTION OF THE DRAWINGS

By way of example only, embodiments of the invention are described with reference to the accompanying drawings, in which:

FIG. 1 shows an exemplary longitudinal section of a tip suitable for a laser operated micro-porator;

FIG. 2 shows an exemplary surface of the tip;

FIG. 3 shows a perspective view of an exemplary tip;

FIG. 4 shows a longitudinal section of a further embodiment of a tip along (B-B);

FIG. 5 shows a cross section along (A-A) of the tip of FIG. 4;

FIG. 6 shows a longitudinal section of a further embodiment of a tip along (B-B);

FIG. 7 shows a cross section along (A-A) of the tip of FIG. 6;

FIG. 8 shows a longitudinal section of a further embodiment of a tip along (B-B);

FIG. 9 shows a cross section along (A-A) of the tip of FIG. 8;

FIG. 10 shows the front end of a longitudinal section of a further tip;

FIG. 11 shows a front view of a tissue biasing element 8a of a further tip;

FIG. 12 shows a perspective view of the tissue biasing element 8a according to FIG. 11;

FIG. 13 shows a front view of a tissue biasing element 8a of a further tip;

FIG. 14 shows a front view of a tissue biasing element 8a of a further tip;

FIG. 15 shows an intersection of the element of FIG. 11 in detail;

FIG. 16 shows a schematic cross-section of two pores of a laser porated skin;

FIG. 17 shows a laser porator;

FIGS. 18a and 18b show a plan view of the skin with an array of microporations;

FIGS. 19 and 20 show a longitudinal section of two further embodiments of a tip;

FIGS. 21 and 22 show detailed structures of tissue biasing elements;

FIG. 23 shows a front view of the tip according to FIG. 19;

FIG. 24 shows a laser porator with a coupled tip;

FIG. 25 shows a further laser porator with a coupled tip;

FIG. 26 shows a top view of a rectangular tip;

FIG. 27 shows a longitudinal section of the rectangular tip of FIG. 26;

FIG. 28 shows a front view of a tip;

FIG. 29 shows a longitudinal section of the tip according to FIG. 28;

FIGS. 30 to 33 show a longitudinal section of various tips pressed onto the skin;

FIG. 34 shows a longitudinal section of a tip pressed onto a finger;

FIGS. 35a to 35d show an actively controlled hollow member 10i in different positions.

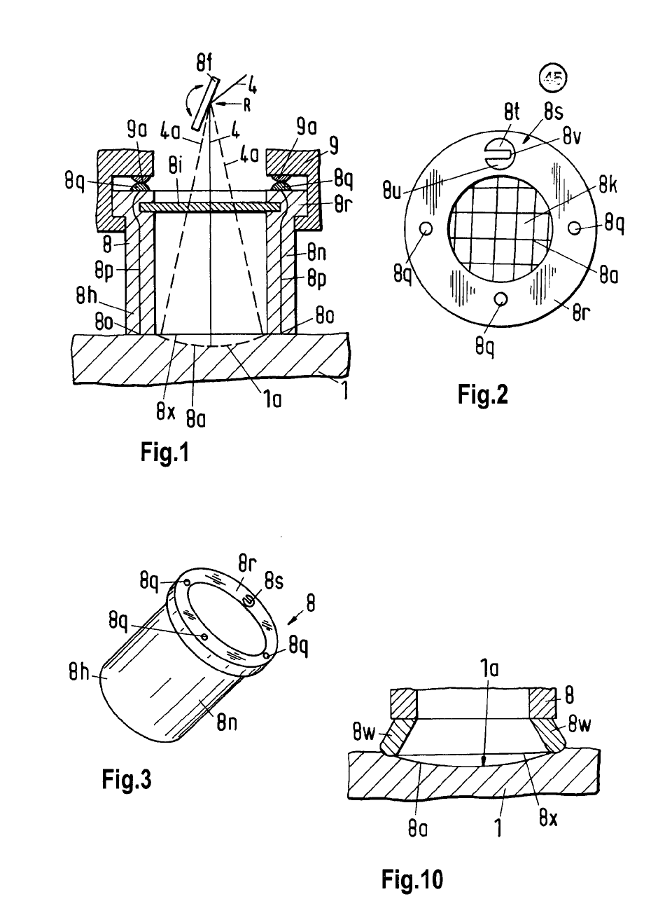

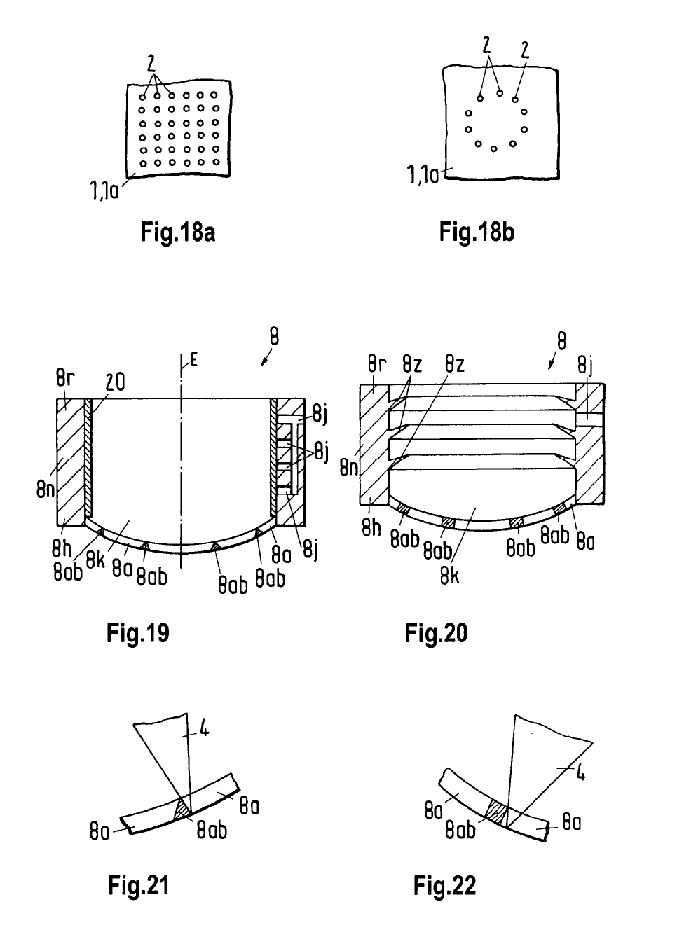

FIG. 1 shows a tip 8 coupled to laser housing 9 of a laser-porator 10, wherein the tip 8 is positioned proximal to the ablation site. The laser-porator 10 comprises at least one swivel-mounted deflecting mirror 8f to deflect a laser beam 4, 4a into various directions onto the skin or biological tissue 1. The tip 8 is for example built as a tubular member comprising a first portion 8r that is configures to be removably coupled to the laser porator 10, and comprising a second portion 8h defining an aperture 8x and comprising a tissue biasing element 8a that is configures to deform at least a portion la of the tissue 1 that is subject to a laser treatment. The tip 8 for a laser emitting device has an optical pathway for the laser beam 4, the laser beam 4 entering the tip 8 at the first portion 8r and exiting the tip 8 at the tissue biasing element 8a. The diameter of the aperture 8x is preferably between 10 mm and 40 mm. The diameter of the laser beam 4 is preferably between 100 μm and 1 mm, which means much smaller than the diameter of the aperture 8x, thus allowing the laser porator 10 to create a plurality of individual pores 2 within the tissue 1 covered by the aperture 8x, by deflecting the laser beam 4,4a accordingly, as disclosed in FIG. 1. By example, FIGS. 18a and 18b disclose two geometrical arrangements of pores 2 created in the skin 1 by placing the tip 8 onto the skin 1, as disclosed in FIG. 1, and by activating the laser porator 10 to create a plurality of individual pores by triggering and by deflecting the laser beam by deflecting mirror 8f.

The tip 8 preferably forms a container with a cylindrical wall 8n and a protective glass 8i. This container collects the ablated tissue and other matter released by the ablation. The tip 8 is preferably shaped so as to allow easy attachment and removal of the tip 8 from the housing 9 of the laser-porator.

The protective glass 8i is an at least partially transparent medium for the laser beam 4 and may be made of glass, polycarbonate, or another medium that is at least partial transparent for the laser beam 4. Instead of the protective glass 8i an optical-path-correction element such as for example a F-Theta lens may be arranged. The use of the optical-path-correction element is advantageous in conjunction with the scanning mirror 8f to create a similar pattern of the laser beam 4 on the skin 1, independent of the deflection of the scanning mirror 8f and independent whether the tissue is cambered to the direction of the laser aperture (like e.g. a toe nail) or it is cambered into the tissue. In a further embodiment a tip 8 comprising an optical-path-correction element as well as a tissue biasing element 8a may be used to ensure the desired shape and position for the laser beam 4 to hit the surface 1a of the skin 1.

The tip 8 comprises a tissue biasing element 8a which biases the portion of skin 1 that is within the opening of the tip 8, which means within the aperture 8x or the optical pathway of the laser beam 4 respectively, into a predetermined shape (e.g., downward, to create a bowl shape) as indicated by the line 1a. Preferably, the tissue biasing element 8a creates a concave shape, in particular a spherical shape, having its center of curvature at point R of the deflecting mirror 8f, wherein Point R is the reflecting point of laser beam 4. Therefore, tissue biasing element 8a will allow laser beam 4 to have about equal length between the deflecting mirror 8f and the surface 1a of the skin 1, which in turn allows creating a highly reproducible geometry of the pores within the skin. Most preferably the tissue biasing element 8a is protruding to the aperture 8x or the optical pathway of the laser beam 4 respectively, as disclosed for example in FIGS. 19 and 20, which means that at least part of the tissue biasing element 8a is arranged in the optical pathway of the laser beam 4, and therefore also might be hit by the laser beam 4. In one preferred embodiment the laser porator 10 triggers and deflects the laser beam 4 such that the laser beam 4 doesn't hit the tissue biasing element 8a but passes the tissue biasing element 8a in the intermediate space defined by the tissue biasing element 8a, as disclosed for example in FIG. 5.

The tip 8 may further comprise electrical contact elements 8o, 8q that are electrically coupled to an electrical conductor such as a wire 8p. The contact elements 8q are connected with the contact elements 9a of the laser device 9. This arrangement allows measuring various physiological parameters (e.g., impedance of the skin 1 between the contact elements 8o) of the skin. Most preferably, the contacts may also be used in a locking mechanism to ensure that the tip 8 is properly positioned on the skin, before the laser source is activated. The tip 8 can comprise further sensors, for example, sensors to measure humidity, temperature, or pH of the skin. Because laser beam 4 might cause injuries if not handled properly, it is important that the laser beam 4 is only activated when the tip 8 is placed onto the skin. Thus, as shown in FIG. 2 and FIG. 3, the disposable tip 8 can include a safety mechanism 8s which allows using the tip 8 only once. The safety mechanism 8s may comprise two contact elements 8t, 8u, with mating contacts in the laser housing 8l, and a fusing element 8v that evaporates after a current has been applied, or breaks mechanically, or is an electronic device (e.g., a microchip), which can be reprogrammed. After poration is finished, a change is applied to the safety mechanism 8s such as burning a fuse element. The status of the safety mechanism 8s is controlled by the laser porator 10 so that the tip 8 can only be used once. Because the laser porator doesn't allow using the same tip twice, this guarantees that a used tip can not be used again by a second person. This also guarantees that a possibly contaminated tip, which was used for poration of the tissue of a first person, can not be used by the second person.

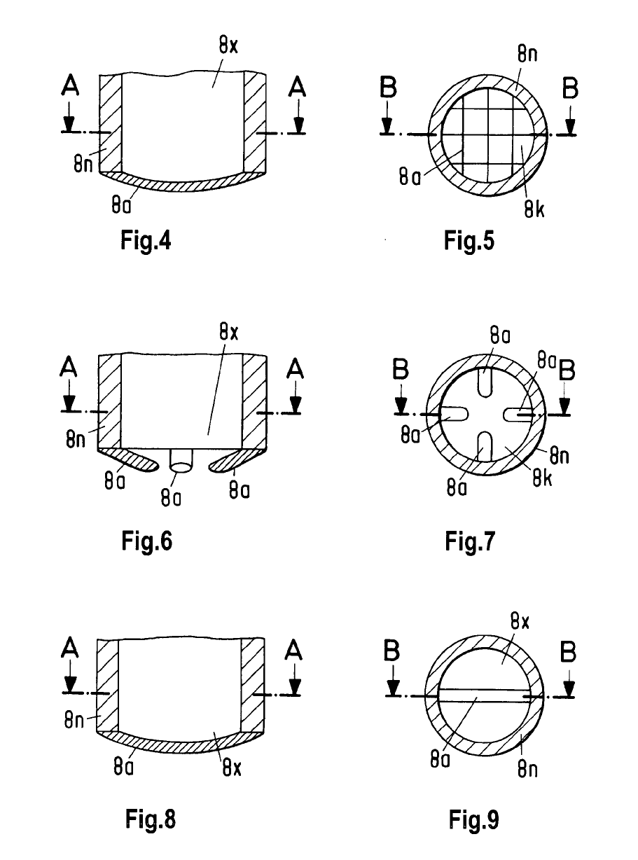

The tissue biasing element 8a of the tip 8 shown in FIG. 4 is a mesh, which may be fabricated from numerous materials, including metal, metal alloys, polymers, glass, plastic, and all reasonable combinations thereof. The exemplary cross section (A-A) illustrated in FIG. 5 shows that the wires or bars are spaced apart to leave an intermediate space in which the laser beam 4 may irradiate the skin surface 1a. In another embodiment, the exemplary tissue biasing element 8a of the tip 8 depicted in FIG. 6 comprises four projecting pins 8a. The cross section (A-A) depicted in FIG. 7 shows the four projecting pins 8a leaving an intermediate space between the projecting pins 8a as well as in the centre. Alternatively, as depicted in FIG. 8, the tissue biasing element 8a of the tip 8 comprises one projecting pin 8a. The cross section (A-A) illustrated in exemplary FIG. 9 shows the projecting pins 8a leaving two intermediate spaces between the projecting pin 8a and the side wall 8n.

It is generally contemplated that the disposable tips 8 as shown in one of the FIGS. 1 to 9 are removably coupled to the housing of the laser-porator. Most preferably, the laser beam 4 is triggered and deflected so that the tissue biasing element 8a is not hit by the laser beam 4 but that only the skin surface 1a is hit. In another preferred embodiment, the laser-porator comprises a reader or detector to read or detect at least one of shape, structure and orientation of the tissue biasing element 8a, to deflect and trigger the laser beam 4 such as to hit only the intermediate spaces of the tissue biasing element 8a. In such devices, the biasing element may have a identifier (e.g., bar code, reflective element, electronic circuit, memory) that provides directly or indirectly information to the porator to identify the tip 8 to the porator.

The tip 8 may further comprise one or more elements 8w to stretch the skin 1, for example, an elastic ring as shown in FIG. 10. When the tip 8 is pressed onto the skin 1, the elastic ring pushed the skin 1 outward in radial direction, so that the skin within the area enclosed by the elastic ring is stretched. Thus, the surface of the skin is pulled tight on the tissue biasing element 8a. The tissue biasing element 8a may be flexible or rigid.

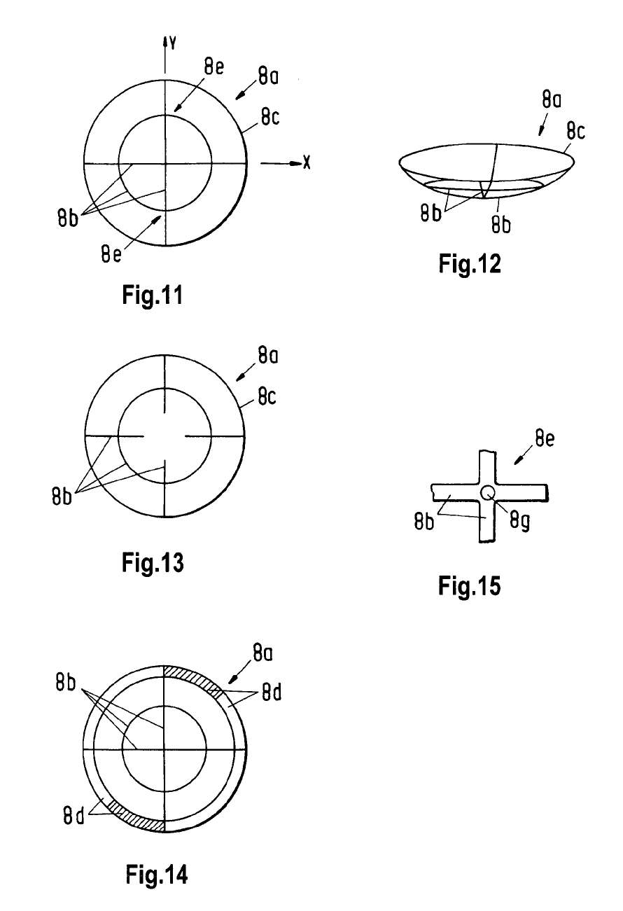

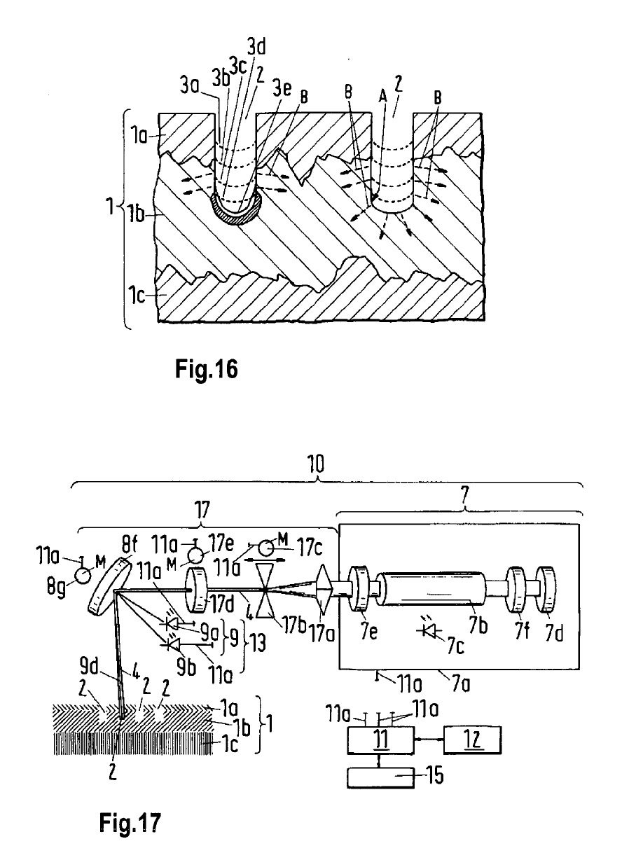

FIG. 11 shows a front view and FIG. 12 a perspective view of a further tissue biasing element 8a having the shape of a partly hemispherical mesh, and comprising elastic material (e.g., metallic wire or plastic element), or comprising a rigid material (e.g., metal or plastic material). The tissue biasing element 8a includes metallic wires 8b connected with an outer ring 8c (preferably made from the same material as the element 8b). As appropriate, the outer ring 8c may be attachable to the cylindrical wall 8n, or may be integral part of the tip 8. Preferably the tissue biasing element 8a has a constant, identical (e.g. spherical or elliptical) curvature. Such a tissue biasing element 8a is in particular advantageous in combination with a single deflection mirror as disclosed in FIGS. 1 and 17. In a further embodiment the tissue biasing element 8a may have different curvatures. For example referring to FIG. 11, the curvature in direction X may be different from the curvature in direction Y, and the tissue biasing element 8a may, for example have a larger radius of curvature in direction X than in direction Y. Such a tissue biasing element 8a is in particular advantageous in combination with a laser porator 10 comprising for example two separate deflection mirrors to deflect the laser beam 4. The focus points of the laser beam deflected by such a deflector arrangement vary depending on the deflection, and are therefore not distributed on a spherical shape. The tissue biasing element 8a having different curvatures allows for example correcting this effect in that the tissue biasing element 8a having such a shape to force the tissue 1 to be treated into a predetermined geometry, such that the laser beam hits the tissue 1 in its focus point.

FIG. 13 shows a front view of an exemplary tissue biasing element 8a leaving an intermediate space in its center. FIG. 14 shows a front view of an exemplary tissue biasing element 8a having two sensors 8d or deflectors 8d arranged on the outer ring 8c (the sensors 8d may be electrically coupled via wires 8p).

It should further be appreciated that various types of connectors (e.g., snap lock or threaded) are suitable to coupled the tip 8 with the housing 9. In one preferred embodiment, the position of the tip 8 with respect to the housing 9 is checked before the laser-porator is directed onto the skin. In another preferred embodiment, the tip 8 comprises an indicator 8g which allows detecting the position of the tip 8 with respect to the housing 9. The indicator 8g can be a reflective surface on the tissue biasing element 8a, which may be arranged on the cross section 8e of the elements 8b as illustrated in FIG. 15. The orientation of the indicator 8g can be detected with a sensor 11b of the laser porator 10 or with the laser beam 4 in combination with a sensor. The indicator 8g may be a reflective area. In a further preferred embodiment, the indicator 8g may be used as safety mechanism 8s in which the properties of the indicator 8g are altered when the tip 8 is used. For example, the laser beam 4 may be directed onto the indicator 8g after porating the skin, to alter or destroy a small reflective layer forming the indicator 8g. Before starting porating the skin, a controller of the laser porator may, by using the laser beam 4 or another sensor, check the status of the indicator 8g, and depending on properties of the indicator 8g, permit or deny poration.

FIG. 17 shows a laser micro-porator 10 comprising a Q-switched or short pulsed laser source 7 and a laser beam shaping and guiding device 17. The laser source 7 has a light source 7c for optical excitation of a laser active material 7b, and a set of reflecting mirrors 7d, 7e. The laser source 7 comprises a laser cavity 7a containing a laser crystal 7b, preferably Er and optional additionally Pr doped YAG, which is pumped by an exciter 7c, the exciter 7c being a single emitter laser diode or a set of single emitter laser diode arrays like emitter bars or stacks of emitter bars. The laser source 7 further comprising an optical resonator comprised of a high reflectance mirror 7d positioned posterior to the laser crystal 7b and an output coupling mirror 7e positioned anterior to the laser crystal 7b, and a saturable absorber 7f positioned posterior to the laser crystal. The saturable absorber 7f works as a Q-switch. A focusing lens 17a and a diverging lens 17b are positioned beyond the output coupling mirror 7e, to create a parallel or quasi-parallel laser beam 4 or a focused laser beam 4. Instead of the lenses 17a, 17b, the microporator 10 could comprise different optical means 17a, 17b, which, for example, focus the laser beam 4 onto the surface of the skin 1. The diverging lens 17b can be moved by a motor 17c in the indicated direction. This allows a broadening or narrowing of the laser beam 4, which allows changing the width of the laser beam 4 and the energy fluence of the laser beam 4. A variable absorber 17d, driven by a motor 17e, is positioned beyond the diverging lens 17b, to vary the energy fluence of the laser beam 4. A deflector 8f, a mirror, driven by an x-y-drive 8g, is positioned beyond the absorber 17d for directing the laser beam 4 in various directions, to create individual pores 2 on the skin 1 on different positions. A control device 11 is connected by wires 11a with the laser source 7, drive elements 17c, 17e, 8g, sensors and other elements not disclosed in detail.

In a preferred embodiment the laser porator 10 also includes a feedback loop 13 respectively a feedback mechanism. In FIG. 17, the feedback loop 13 comprises an apparatus 9 to measure the depth of the individual pore 2, and preferably includes a sender 9a with optics that produce a laser beam 9d, and a receiver with optics 9b. The laser beam 9d has a smaller width than the diameter of the individual pore 2, for example five times smaller, so that the laser beam 9d can reach the lower end of the individual pore 2. The deflection mirror 8f directs the beam of the sender 9a to the individual pore 2 to be measured, and guides the reflected beam 9d back to the receiver 9b. This distance measurement device 9, which can be built in different way, allows measuring the position of the lower end e.g. the depth of the individual pore 2. In a preferred embodiment, the depth of the individual pore 2 is measured each time after a pulsed laser beam 4 has been emitted to the individual pore 2, allowing controlling the effect of each laser pulse onto the depth of the individual pore 2. The feedback loop 13 can be built in various ways to be able to measure a feedback signal of an individual pore 2. The feedback loop 13 may, for example, comprise a sender 9a and a receiver 9b, built as a spectrograph 14, to detect changes in the spectrum of the light reflected by the lower end of the individual pore 2. This allows, for example, detecting whether the actual lower end 3a, 3b, 3c, 3d of the individual pore 2 is part of the stratum corneum 1a or of the epidermis 1b. The laser porator 10 also comprises a poration memory 12 containing specific data of the individual pores 2, in particular the initial microporation dataset. The laser porator 10 preferably creates the individual pores 2 as predescribed in the poration memory 12. The laser porator 10 also comprises one or more input-output device 15 or interfaces 15, to enable data exchange with the porator 10, in particular to enable the transfer of the parameters of the individual pores 2, the initial microporation dataset, into the poration memory 12, or to get data such as the actual depth or the total surface Ai of a specific individual pore 2i. The input-output device 15 can be a card reader, a scanner, a wired interface or for example a wireless connection such as Bluetooth.

The porator further can comprise one or more input-output devices or user interfaces 15 for manually exchange date like data of substances, individuals and much more. The user interface can for example comprise displays, buttons, voice control or a finger print sensor.

There are different ways to build a laser source 7. The laser source 7 may, for example, be built as a laser diode with optics that create a beam 4 of fixed width, for example a width of 250 μm.

The pulse repetition frequency of the laser source 7 is within a range of 1 Hz to 1 MHz, preferably within 100 Hz to 100 kHz, and most preferred within 500 Hz to 10 kHz. Within one application of the laser porator 10, between 2 and 1 million individual pores 2 can be produced in the biological membrane 1, preferably 10 to 10000 individual pores 2, and most preferred 10 to 1000 individual pores 2, each pore 2 having a width in the range between 0.05 mm and 0.5 mm or up to 1 mm, and each pore 2 having a depth in the range between 5 μm and 200 μm, but the lower end of the individual pore 2 being preferably within the epidermis 1b. If necessary the porator 10 is also able to create pores of more than 200 μm depth.

The laser porator 10 also comprises an interlock mechanism, so that a laser pulse is emitted only when it is directed onto the skin 1. The feedback loop 13 could for example be used to detect whether the pulse is directed onto the skin 1. Those skilled in the art will appreciate that there are numerous ways to create an interlock mechanism, and all such ways are contemplated. One embodiment is described in FIG. 4a.

FIG. 17 discloses a circular laser beam 4 creating a cylindrical individual pore 2. The individual pore 2 can have other shapes, for example in that the laser beam 4 has not a circular but an elliptical shape, a square or a rectangle. The individual pore 2 can also be shaped by an appropriate movement of the deflector 8f, which allows creation of individual pores 2 with a wide variety of shapes.

FIG. 18a shows a plan view of the skin having a regular array of individual pores 2 that collectively form a micro-poration. The micro-poration on the biological membrane, after the laser porator 10 has finished porating, is called “initial microporation”. The poration memory 12 preferably contains the initial microporation dataset, which define the initial microporation. The initial microporation dataset comprises any suitable parameters, including: width, depth and shape of each pore, total number of individual pores 2, geometrical arrangement of the pores 2 on the biological membrane, minimal distance between the pores 2, and so forth. The laser porator 10 creates the pores 2 as defined by the initial microporation dataset. This also allows arranging the individual pores 2 in various shapes on the skin 1, as for example disclosed with FIG. 18b.

FIGS. 19 and 20 disclose a longitudinal section of two further tips 8 comprising a tubular body 8n which has a first portion 8r and a second portion 8h defining an aperture 8x and having the shape of a tubular channel, and comprising a biasing element 8a. The biasing element 8a having a grid like structure as disclosed in FIG. 5. The tips 8 have no protective glass 8i, but may in an advantageous embodiment also comprise a protective glass 8i or an optical-path-correction element such as for example a F-Theta lens 8i arranged between the first portion 8r and the second portion 8h. Both tips 8 disclosed in FIGS. 19 and 20 comprise a suction aperture 8j which allows removing gas and debris from within the tip 8. The tip 8 disclosed in FIG. 19 comprises a plurality of suction apertures 8j arranged on the inner wall of the tubular body 8n. In a preferred embodiment the tip 8 comprises a filter 20 arranged in front of the inner wall of the tubular body 8n, to prevent biological material from leaving the inner space of the tip 8, whereby this biological material is preferably deposited in the filter 20. The filter 20 may be arranged in various ways in the tip 8. For example the filter 20 may also be arranged within the suction aperture 8j. Instead of a filter 20, the embodiment disclosed in FIG. 20 comprises three collecting elements 8z arrange one above the other along the inner side wall of the tubular body 8n. The collecting element 8z may protrude into the inner space of the tip 8, as disclosed in FIG. 20. But the collecting element 8z may also be arranged within the wall of the tubular body 8n, the inner wall of the tubular body 8n having a recess to form the collecting elements 8z. The purpose of these collecting elements 8z is also to collect debris of the biological tissue 1. In one embodiment the tip 8 according to FIG. 20 comprises no suction aperture 8j. In a preferred embodiment, the tip 8 also comprises a suction aperture 8j, and also may comprise a filter 20, for example arranged within the suction aperture 8j. FIG. 19 discloses a tissue biasing elements having bars 8ab of triangular shape, whereas FIG. 20 discloses a tissue biasing element 8a having bars 8ab of rectangular shape.

FIG. 21 shows in detail a triangular bar 8ab of FIG. 19, whereas FIG. 22 shows in detail a rectangular bar 8ab of FIG. 20 in detail. As disclosed in FIGS. 21 and 22 the triangular shape of the bar 8ab has the advantage that a laser beam 4 directed onto the biological tissue 1 may be closer to the bar 8ab, compared with a rectangular bar 8ab, where the laser beam 4 is not able to irradiate the biological tissue 1 just beside the bar 8ab. There is a gap between the bar 8ab and the irradiation point of laser beam 4. The laser beam 4 can not irradiate biological tissue 1 arranged just below the bars 8ab. The triangular bars 8ab, or more generally bars 8ab having a non-rectangular shape, in particular the bars 8ab having a cross sectional shape which is increasing in longitudinal direction E as disclosed in FIG. 19, have the advantage to reduce the area of the biological tissue 1 which is not accessible for the laser beam 4. The tips according to FIGS. 19 and 20 are preferably produced by injection moulding.

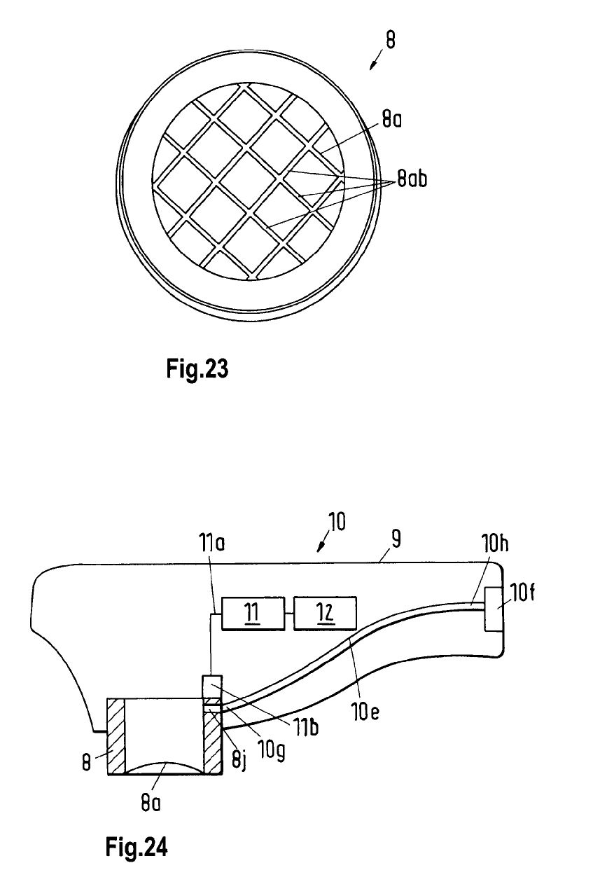

FIG. 23 shows a front view of the tip 8 disclosed in FIG. 19. The tip 8 comprises a biasing element 8a, having a grid like structure with a plurality of bars 8ab. In further advantageous embodiments the biasing element 8a may have the form of honeycombs or a spiral.

The tissue biasing element 8a may also have a concave shape, as disclosed in FIG. 24, or may also have a planar shape.

FIG. 24 discloses a laser porator 10 comprising a housing 9 and a tip 8 removably coupled to the housing 9. The laser porator 10 may comprise the elements disclosed in FIG. 17. In a preferred embodiment the laser porator 10 may comprise a fluid conduct 10e comprising a first end 10g arranged adjacent the suction aperture 8j of a coupled tip 8, and comprising a second end 10h which is connected to a means for fluid suction, such as a ventilator 10f.

In a preferred embodiment the laser porator 10 comprises sensors 11b or communication means 11b to detect or read at least one of position, shape, type and use of the tip 8 and/or the tissue biasing element 8a. In a preferred embodiment the laser porator 10 comprises a memory 12 to store data of the geometry of the tissue biasing element 8a, in particular the position of the bars 8ab. Preferably the laser porator 10 comprises a control device 11 that deflects by controlling deflector 8f the laser beam based on characteristics of the of the tissue biasing element 8a, in particular to avoid that the laser beam 4 hits the tissue biasing element 8a. In a preferred mode of operation the control device 11 deflects the laser beam 4 by controlling the position of the mirror 8f and by triggering the laser beam 4 in such a way that no laser beam 4 hits the tissue biasing element 8a.

In a simpler embodiment, the laser porator 10 doesn't consider the position of the tissue biasing element 8a, and in particular the bars 8ab, leading to the effect that the laser beam 4 may hit the tissue biasing element 8a, which causes that disadvantage the no pore will be created in the biological tissue 1 on this specific position. This is of no disadvantage if the exact total number and/or shape of the created pores 2 is not of importance.

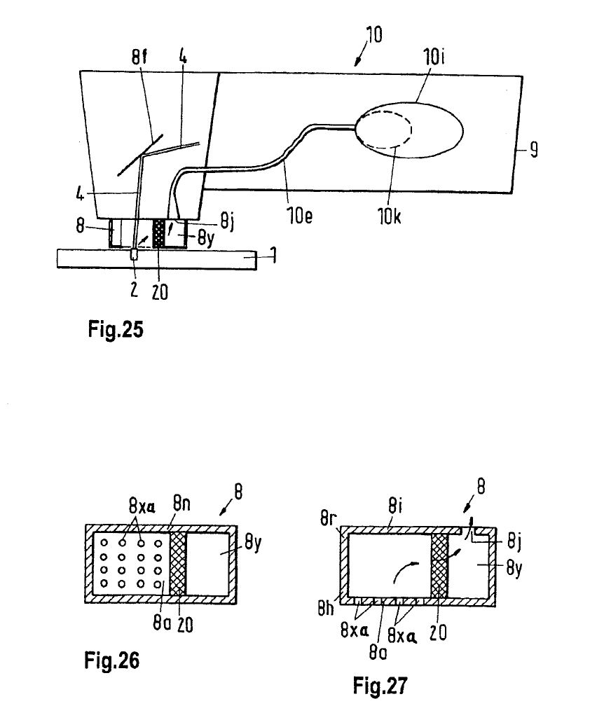

FIG. 25 discloses a further embodiment of a laser porator 10 comprising a housing 9 and a tip 8 removable coupled to the housing 9. The laser porator 10 may comprise the elements disclosed in FIG. 17, of which only the deflector 8f and the laser beam 4 are shown in FIG. 25. The porator 10 comprises a fluid conduct 10e connecting an aperture 8j of the tip 8 with an elastic hollow member 10i. The hollow member 10i may be connected with a drive, which is not shown, to vary the volume of the hollow member 10i, as disclosed, for example by the reduced volume 10k. When starting ablation by the laser beam 4 the volume 10k may grow to the volume 10i to thereby suck in fluid of the tip 8. The tip 8 comprises a second cavity 8y separated from the cavity where the laser beam 4 passes, but fluidly connected with each other. FIG. 26 shows a top view of the tip 8 disclosed in FIG. 25. The tip 8 is build as a rectangular member 8n comprising on the left a first cavity though which the laser beam 4 passes. The tip 8 comprises to the right a second cavity 8y, which is separated but fluidly connected with the first cavity where the laser beam 4 passes. The two cavities may be fluidly connected by an aperture, by a permeable membrane 20, in particular a semi permeable membrane, or by a filter 20. It might be advantageous that the material ablated by the laser beam 4 is kept within the tip 8. In a preferred embodiment the tip 8 is built such that the ablated material is kept as fare as possible in the second cavity 8y. The tip 8 may, for example be provided with a semi permeable membrane 20 and a filter 20 arranged in front of the aperture 8j, to keep the ablated material within the second cavity 8y.

To keep ablated material within tip 8, it might also be advantageous that the tip 8 comprises a tissue biasing element 8a with a plurality of apertures 8x, though which the laser beam 4 passes to hit the underlying tissue 1. The apertures 8x support to keep the ablated material within the first cavity. FIG. 27 shows a longitudinal section of the rectangular tip 8 disclosed in FIG. 26. The tip 8 comprises a protective glass 8i, so the first cavity is closed with the exception of the apertures 8x. It might be advantageous to detain the ablated material within the tip 8. A filter 20 has been arranged between the first cavity and the second cavity 8y to collect ablated material. The thereby filtered fluid leaves the second cavity 8y by the aperture 8j.

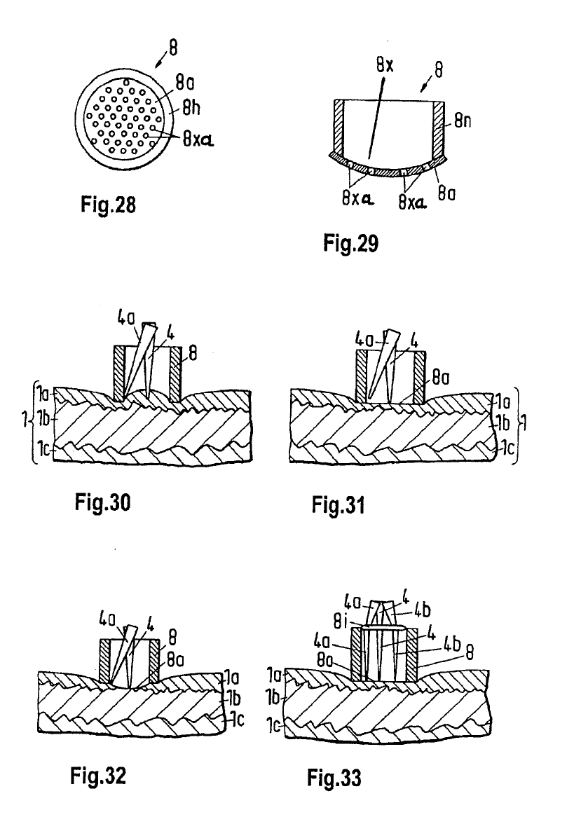

FIG. 28 shows a front view of a further tip 8 comprising a tissue biasing element 8a with a plurality of apertures 8x through which the laser beam 4 has to pass, to hit the tissue 1. FIG. 29 shows a longitudinal section of the tip according to FIG. 28. The tip 8 disclosed in FIGS. 28 and 29 may also comprise a protective glass 8i as for example disclosed in FIG. 1, to detain the ablated material within the tip 8.

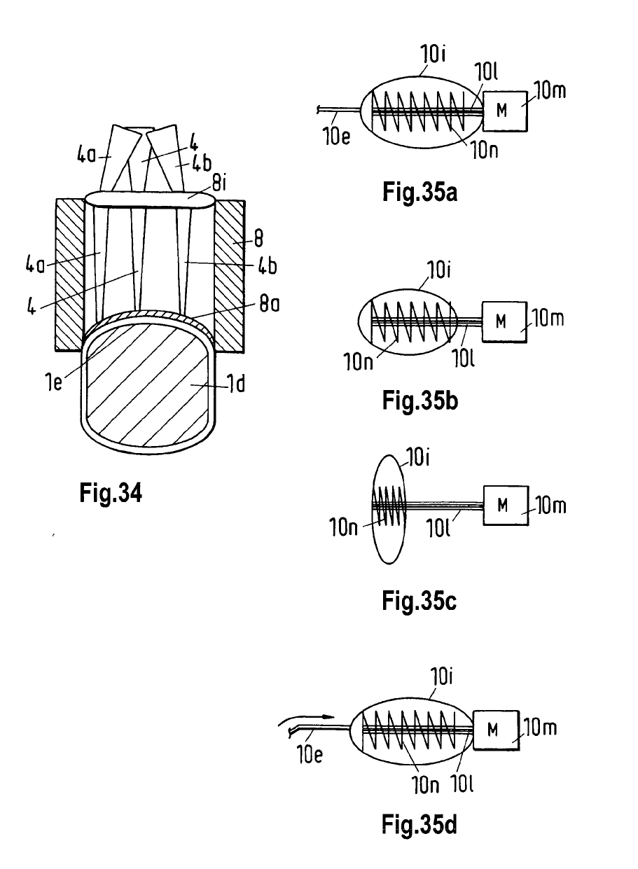

FIG. 30 shows a tip 8 pressed onto the skin 1, the tip 8 having no tissue biasing element 8a. The disadvantage of such a tip 8 is that the laser beam 4 and the deflected laser beam 4a hit the skin 1 not with the same focus. The laser beams 4, 4a therefore hit the skin 1 with different intensity, which causes pores 2 of different shapes and properties. FIG. 31 shows a tip 8 pressed onto the skin 1, the tip 8 having a planar tissue biasing element 8a. Such a tip 8 used in combination with a deflectable laser beam 4,4a also leads to the disadvantage that the laser beams 4, 4a hit the skin 1 with different intensity. FIG. 32 shows a tip 8 pressed onto the skin 1, the tip 8 having a convex tissue biasing element 8a. Most preferably the curvature of the tissue biasing element 8a is adapted to deflect the laser beam 4,4a such that preferably all deflected laser beams 4,4a,4b, . . . hit the skin 1 at about the same point of focus, which allows to hit the skin 1 with a laser beam 4 or a laser pulse with similar energy. This allows creating a plurality of pores 2 with reproducible shape and properties. FIG. 33 shows another tip 8 pressed onto the skin 1. Tip 8 comprises a planar tissue biasing element 8a as well as an F-Theta lens 8i. As disclosed the F-Theta lens 8i causes the various deflected laser beams 4, 4a, 4b to hit the skin at a defined point of focus. FIG. 34 shows another tip 8 pressed onto the finger nail 1e of a finger 1d. The tip comprises a concave tissue biasing element 8a adapted to the shape of the finger nail. The tip 8 comprises an optical path correction element 8i which is adapted such that the deflected laser beams 4, 4a, 4b hit the finger nail 1e with their focal point. A man skilled in the art understands how to adopt and choose an optical path correction element (for example shape, refraction index, thickness and so on) such that the deflected laser beam 4,4a,4b is focused on the tissue hit by the laser beam 4,4a,4b, also when the tissue biasing element 8a has a planar, convex or concave shape.

The optical path correction element 8i may be part of the tip 8, but most preferably the optical path correction element 8i is part of the laser porator 10, so the same optical path correction element 8i can be used many times.

FIGS. 35a to 35d show an actively controlled hollow member 10i in different positions, the hollow member 10i being preferably arranged within the housing 9 of the ablator 10 and being connected by line 10e with the aperture 8j of tip 8. Before using the ablator 10, a motor 10m comprising a shaft 10l compresses a spring 10n, whereby the hollow member 10i is connected to the spring On such that the volume of the hollow member 10i decreases, as disclosed in FIGS. 35b and 35c. Having reached the end position, as disclosed in FIG. 35c, the ablator 10 starts ablating the tissue 1 using laser beam 4. Preferably after or shortly before terminating the ablation, the spring 10n is released so that the hollow member 10i expands to the original shape and volume, as disclosed in FIG. 35d. The increase in volume of the hollow member 10i causes a fluid to flow along line 10e to within the hollow member 10i. Most advantageously spring 10n is expanded very fast, to thereby expand the hollow member 10i within for example fractions of a second, thereby causing a high and abrupt suction of the fluid from within tip 8. One advantage of the abrupt suction of the fluid form tip 8 is that most of the ablated material is affected. In an advantageous embodiment tip 8 comprises a filter 20 as disclosed in FIG. 25, and the abrupt suction of the fluid from within the first cavity causing most of the ablated material to be caught by filter 20, to thereby advantageously leaving little or no ablated material within the first cavity, and to thereby advantageously leaving little or no ablated material within the second cavity 8y.

Thus, specific embodiments and applications of tips for a laser emitting device for porating of biological tissue or skin have been disclosed. It should be apparent, however, to those skilled in the art that many more modifications besides those already described are possible without departing from the inventive concepts herein. The inventive subject matter, therefore, is not to be restricted except in the spirit of the present disclosure. Moreover, in interpreting the specification and contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein, is inconsistent or contrary to the definition of that term provided herein, the definition of that term provided herein applies and the definition of that term in the reference does not apply.

It should be especially appreciated that the tip 8 described herein is configured to be used in combination with a laser porator. Therefore, it should be recognized that such tips may not only be used for treating skin related conditions but may also be used independently in applications where microporation, and particularly microporation with predetermined pore geometry or drug delivery kinetic/dynamic is required. For example, contemplated alternative uses include application of the tip to create pores for systemic, transdermal administration of permeants and drugs, such as the administration of high amount of drugs, and also the transdermal administration through an area of equal or less than 1 cm².

1. A tip (8) comprising a first portion (8r) that is configured to be removably coupled to a laser porator (10) and a second portion (8h) defining an aperture (8x), wherein the second portion (8h) comprises a tissue biasing element (8a) that is configured to deform at least a portion of a tissue that is subject to a laser treatment, and wherein the aperture (8x) defines a pathway for the laser beam, and wherein the tissue biasing element (8a) is configured to at least partially protrude into the pathway.

2. (canceled)

3. The tip of claim 1, wherein the tip has an optical pathway for the laser beam (4), and wherein the tissue biasing element (8a) is protruding into the optical pathway.

4. The tip of claim 1 wherein the tissue biasing element (8a) is configured such that the average distance between the deformed tissue and a laser steering mirror (8f) of the laser porator (10) is substantially the same.

5. The tip of claim 4 wherein the average distance is the focal distance of the laser beam.

6. The tip of claim 1 wherein the tissue biasing element (8a) deforms the tissue in one of a concave manner, a convex manner, and a flat manner.

7. (canceled)

8. (canceled)

9. The tip of claim 1 wherein the tissue biasing element (8a) has different curvatures.

10. The tip of claim 1 wherein the tissue biasing element (8a) has a form selected from the group consisting of a wire frame, centrally protruding elements, honeycomb, mesh and a spiral.

11. The tip of claim 1 wherein the tip has a suction aperture (8j).

12. The tip of claim 11 having a plurality of suction apertures (8j) arranged on the inner surface of a tubular member (8n).

13. The tip of claim 11 wherein a filter (20) is arranged in front of the suction aperture (8j).

14. The tip of claim 1 wherein a least one collecting element (8z) is arranged between the first portion (8r) and the second portion (8h).

15. The tip of claim 14, wherein the collecting element (8z) is protruding into an inner space of the tip.

16. (canceled)

17. The tip of claim 1, wherein the tissue biasing element (8a) has a cross sectional shape of a triangle or a circle segment.

18. The tip of claim 1 comprising a second cavity 8y separated from a first cavity where the laser beam 4 passes, the first and second cavity being fluidly connected with each other.

19. (canceled)

20. (canceled)

21. The tip of claim 1 wherein an optical-path-correction element (8i) is arranged between the first portion (8r) and the second portion (8h).

22. The tip of claim 1 comprising at least one of a sensor (8h) and a marker (8g).

23. A laser porator (10) adapted to removably couple a tip (8) according to claim 1.

24. The laser porator according to claim 23, including a fluid conduct (10e) comprising a first end (1Og) arranged adjacent the suction aperture (8j) of a coupled tip (8), and comprising a second end (10h) which in connected to a suction device, in particular a ventilator (10f).

25. The laser porator according to claim 23, comprising a sensor (11b) to detect at least one of position, shape, type and use of the tissue biasing element (8a).

26. The laser porator according to claim 23, comprising a memory (12) to store data of the geometry of the tissue biasing element (8a).

27. The laser porator according to claim 23, comprising a control device (11) that deflects the laser beam (4) based on characteristics if the tissue biasing element (8a).

28. The laser according to claim 27, wherein the control device (11) deflects and triggers the laser beam (4) such that no laser beam (4) hits the tissue biasing element (8a).

29. A kit comprising a plurality of tips according to claim 1 and comprising a laser porator (10) according claim 23.

30. The tip of claim 1 wherein the tissue biasing element (8a) has a spherical shape.
